(19) United States
(12) Patent Application Publication
(10) Pub. No.: US 2025/0025066 A1
(43) Pub. Date: Jan. 23, 2025

(54) GAIT MEASUREMENT SYSTEM AND METHOD USING MULTIPLE LIDARS AT DIFFERENT HEIGHTS

(71) Applicant: Medicalsolutionsystem, Gimhae City (KR)

(72) Inventors: Minsoo Kang, Busan (KR); JoonMo Seo, Busan (KR)

(21) Appl. No.: 18/744,529

(22) Filed: Jun. 14, 2024

(30) Foreign Application Priority Data
Jul. 21, 2023 (KR) 10-2023-0094995

(51) Int. Cl. A61B 5/11; G01S 7/48; G01S 17/87; G01S 17/88

(52) U.S. Cl. CPC A61B 5/112; A61B 5/1114; A61B 5/1126; G01S 7/4802; G01S 17/87; G01S 17/88; A61B 2562/04

(57) ABSTRACT

Proposed are a gait measurement system and method using multiple LiDARs of different heights. The system includes a walking path for a measurement subject to walk, a LiDAR sensor device including first, second, and third LiDAR sensors, installed at a side of the walking path, and configured to scan a direction of the walking path, and a gait analysis part configured to use signals detected by the LiDAR sensor device to analyze a gait of the measurement subject walking on the walking path. The method includes collecting scanning data obtained by the LiDAR sensor device scanning the walking path on which the measurement subject is walking, analyzing the scanning data to specify foot positions, knee positions, and a torso position of the measurement subject, and combining the specified positions of the measurement subject to analyze at least one selected from a group of body distortion and gait instability during walking.

### CROSS-REFERENCE TO RELATED APPLICATION

The present application claims priority to Korean Patent Application No. 10-2023-0094995, filed Jul. 21, 2023, the entire contents of which is incorporated herein for all purposes by this reference.

### BACKGROUND OF THE INVENTION

### Field of the Invention

The present disclosure relates to a gait measurement system and method. More particularly, the present disclosure relates to a gait measurement system and method using multiple LiDARs at different heights.

### Description of the Related Art

The description in this section merely provides background information on one embodiment of the present disclosure and does not constitute the related art.

Gait analysis is intended to identify idiosyncrasies in the way a person walks, and measures several aspects of a gait to provide a comprehensive evaluation. More specifically, gait analysis may include kinematic analysis, dynamic electromyography, and energy expenditure measurement. Gait analysis may be applied to understand the characteristics of posture or movement of people with leg lesions and spinal misalignment, or to help athletes run more efficiently.

Currently, gait analysis in rehabilitation medicine measures and analyzes a gait using expensive load-measuring mattresses or high-speed cameras. However, this type of gait measurement requires various sensors to be attached to a subject's body, is expensive, and requires a long preparation time for measurement. In addition, the gait measurement is performed in a controlled environment of a laboratory, so there is a limitation in analyzing a gait in daily life.

In recent years, modern people have been spending more time sitting, which has led to an increase in scoliosis, pelvic distortion, and flat feet, and these problems affect gait. Especially for children and teenagers, it is important to recognize and correct poor posture early and develop good gait habits.

As such, there is a growing need for gait measurement and analysis, but it is difficult to easily access conventional gait analysis methods due to costs and limitations. Therefore, there is a need to develop technologies that make gait analysis easier and less expensive.

In the meantime, as a related art of the present disclosure, Korean Patent No. 10-1895399 (invention title: GAIT ANALYSIS AND CORRECTION APPARATUS OF BASE INFORMATION AND COMMUNICATIONS TECHNOLOGY, registration date: 30 Aug. 2018) is disclosed.

The above-described background technology is technical information that has been possessed by the present inventor in order to derive the present disclosure or which has been acquired in the process of deriving the present disclosure, and can not necessarily be regarded as well-known technology which has been known to the public prior to the filing of the present disclosure.

### SUMMARY OF THE INVENTION

The present disclosure has been made keeping in mind the above problems occurring in the previously proposed methods, and the present disclosure is directed to providing a gait measurement system and method using multiple LiDARs at different heights, wherein a LiDAR sensor device including a first LiDAR sensor for scanning at foot height, a second LiDAR sensor for scanning at knee height, and a third LiDAR sensor for scanning at torso height is installed at a side of a walking path, and signals detected by the LiDAR sensor device are used to analyze a gait of a measurement subject walking on the walking path. Accordingly, it is easy to recognize the movement behavior of the feet, knees, and torso during walking. In addition, through an analysis by combining scanning data of the feet, knees, and torso during walking, information, such as the movement of the subject's center of gravity, required for rehabilitation medicine may be provided, and used for recognizing the use of a cane, scoliosis, or changes in prognosis before and after surgery.

However, the objectives of the present disclosure are not limited thereto, and there may be other objectives. Even if not explicitly mentioned, the objectives or effects that can be understood from the solutions or embodiments are also objectives of the present disclosure.

According to an aspect of the present disclosure, there is provided a gait measurement system using multiple LiDARs at different heights,
the gait measurement system including:
a walking path for a measurement subject to walk;
a LiDAR sensor device installed at a side of the walking path and configured to scan a direction of the walking path; and
a gait analysis part configured to use signals detected by the LiDAR sensor device to analyze a gait of the measurement subject walking on the walking path,
wherein the LiDAR sensor device includes:
a first LiDAR sensor configured to collect first scanning data by scanning, on a floor at the side of the walking path, a plane at foot height of the measurement subject walking on the walking path;
a second LiDAR sensor configured to collect second scanning data by scanning, at the side of the walking path, a plane at knee height of the measurement subject walking on the walking path; and
a third LiDAR sensor configured to collect third scanning data by scanning, at the side of the walking path, a plane at torso height of the measurement subject walking on the walking path.

Preferably, the gait analysis part may be configured to
analyze the first scanning data to specify foot positions of the measurement subject, analyze the second scanning data to specify knee positions of the measurement subject, and analyze the third scanning data to specify a torso position of the measurement subject, and
use the foot positions of the measurement subject to analyze at least one selected from a group of step length, the number of steps, and gait speed, and use the knee positions of the measurement subject to analyze at least one selected from a group of a distance between knees and knee movement speed.

More preferably, the gait analysis part may be configured to
combine the specified foot positions, knee positions, and torso position of the measurement subject to analyze at least one selected from a group of body distortion and gait instability while the measurement subject is walking.

Preferably, a plurality of the LiDAR sensor device may be
installed at intervals of a predetermined distance on the floor at the side of the walking path.

Preferably, a plurality of the LiDAR sensor devices may be
arranged to face each other across the walking path.

Preferably,
the gait measurement system may further include a client terminal on which an application program for outputting an analysis result obtained by the gait analysis part is installed.

According to an aspect of the present disclosure, there is provided a gait measurement method using multiple LiDARs at different heights,
to measure a gait of a measurement subject walking on a walking path, the gait measurement method including the steps, each performed by a gait measurement system including
the walking path for the measurement subject to walk,
a LiDAR sensor device installed at a side of the walking path and configured to scan a direction of the walking path, and
a gait analysis part configured to use signals detected by the LiDAR sensor device to analyze the gait of the measurement subject walking on the walking path, of:
(1) collecting scanning data obtained by the LiDAR sensor device scanning the walking path on which the measurement subject is walking, wherein first scanning data is collected by a first LiDAR sensor scanning, on a floor at the side of the walking path, a plane at foot height of the measurement subject walking on the walking path, second scanning data is collected by a second LiDAR sensor scanning, at the side of the walking path, a plane at knee height of the measurement subject walking on the walking path, and third scanning data is collected by a third LiDAR sensor scanning, at the side of the walking path, a plane at torso height of the measurement subject walking on the walking path;
(2) analyzing the first scanning data to specify foot positions of the measurement subject, analyzing the second scanning data to specify knee positions of the measurement subject, and analyzing the third scanning data to specify a torso position of the measurement subject; and
(3) combining the specified foot positions, knee positions, and torso position of the measurement subject to analyze at least one selected from a group of body distortion and gait instability while the measurement subject is walking.

Preferably, the gait measurement method may further include after the step (3),
(4) providing an analysis result obtained in the step (3) to a client terminal on which an application program for outputting the analysis result obtained by the gait analysis part is installed.

According to the gait measurement system and method using multiple LiDARs at different heights proposed in the present disclosure, a LiDAR sensor device including a first LiDAR sensor for scanning at foot height, a second LiDAR sensor for scanning at knee height, and a third LiDAR sensor for scanning at torso height is installed at a side of a walking path, and signals detected by the LIDAR sensor device are used to analyze a gait of a measurement subject walking on the walking path. Accordingly, it is easy to recognize the movement behavior of the feet, knees, and torso during walking. In addition, through an analysis by combining scanning data of the feet, knees, and torso during walking, information, such as the movement of the subject's center of gravity, required for rehabilitation medicine may be provided, and used for recognizing the use of a cane, scoliosis, or changes in prognosis before and after surgery.

Furthermore, various useful advantages and effects of the present disclosure are not limited to the above-described contents, and will be more easily understood in the process of describing specific embodiments of the present disclosure.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a diagram illustrating the configuration of a gait measurement system using multiple LiDARs at different heights according to an embodiment of the present disclosure;

FIG. 2 is a diagram illustrating communication with a client terminal in a gait measurement system using multiple LiDARs at different heights according to an embodiment of the present disclosure;

FIGS. 3 and 4 are diagrams illustrating gait measurement through a gait measurement system using multiple LiDARs at different heights according to an embodiment of the present disclosure;

FIG. 5 is a diagram illustrating a LiDAR sensor device of a gait measurement system using multiple LiDARs at different heights according to an embodiment of the present disclosure;

FIG. 6 is a diagram illustrating the flow of a gait measurement method using multiple LiDARs at different heights according to an embodiment of the present disclosure;

FIG. 7 is a diagram illustrating gait measurement experiment and scanning data from a gait measurement system using multiple LiDARs at different heights according to an embodiment of the present disclosure;

FIG. 8 is a diagram illustrating a plurality of LiDAR sensor devices arranged in a gait measurement system using multiple LiDARs at different heights according to an embodiment of the present disclosure; and

FIG. 9 is a diagram illustrating a plurality of LiDAR sensor devices of different shapes arranged in a gait measurement system using multiple LiDARs at different heights according to an embodiment of the present disclosure.

### DETAILED DESCRIPTION OF THE INVENTION

Hereinafter, embodiments of the present disclosure will be described in detail with reference to the accompanying drawings such that the present disclosure can be easily embodied by those skilled in the art to which the present disclosure belongs. However, the present disclosure may be embodied in various different forms and should not be limited to the embodiments set forth herein. Further, in order to clearly explain the present disclosure, portions that are not related to the present disclosure are omitted in the drawings, and like reference numerals designate like elements throughout the specification.

Throughout the specification, when a part is referred to as being “connected” to another part, it includes not only being “directly connected”, but also being “indirectly connected” by interposing the other part therebetween. In addition, when a part “includes” an element, it is noted that it further includes other elements, but does not exclude other elements, unless specifically stated otherwise, and is not to be understood as precluding the possibility that one or more other features, numbers, steps, operations, elements, parts, or combinations thereof may exist or may be added.

The embodiments described hereinafter are detailed descriptions of the present disclosure to facilitate understanding of the present disclosure and are not intended to limit the scope of the present disclosure. Thus, subject matter having the same scope and performing the same function as that of the present disclosure also falls within the scope of the present disclosure.

In addition, the elements, processes, steps, or methods included in the embodiments of the present disclosure may be shared as long as they do not technically conflict with each other.

In addition, part of the operations or functions described as being performed by a terminal, apparatus, or device in the present disclosure may be performed instead by a server connected to the terminal, apparatus, or device. Similarly, part of the operations or functions described as being performed by the server may be performed by the terminal, apparatus, or device connected to the server.

In particular, the means for executing a system according to each embodiment of the present disclosure may be an application or a web server. Examples of a terminal that is the means for reading a recording medium on which the application or the web server is recorded may include general PCs, such as general desktop and laptop computers, and mobile terminals, such as smartphones and tablet PCs.

Hereinafter, embodiments of the present disclosure will be described in detail with reference to the accompanying drawings.

FIG. 1 is a diagram illustrating the configuration of a gait measurement system 100 using multiple LiDARs at different heights according to an embodiment of the present disclosure. As shown in FIG. 1, according to an embodiment of the present disclosure, a gait measurement system 100 using multiple LiDARs at different heights includes: a walking path 110 for a measurement subject to walk; a LiDAR sensor device 120 installed at a side of the walking path 110, including three LiDAR sensors 121, 122, and 123, and configured to scan the direction of the walking path 110; and a gait analysis part 130 configured to use signals detected by the LiDAR sensor device 120 to analyze a gait of the measurement subject walking on the walking path 110.

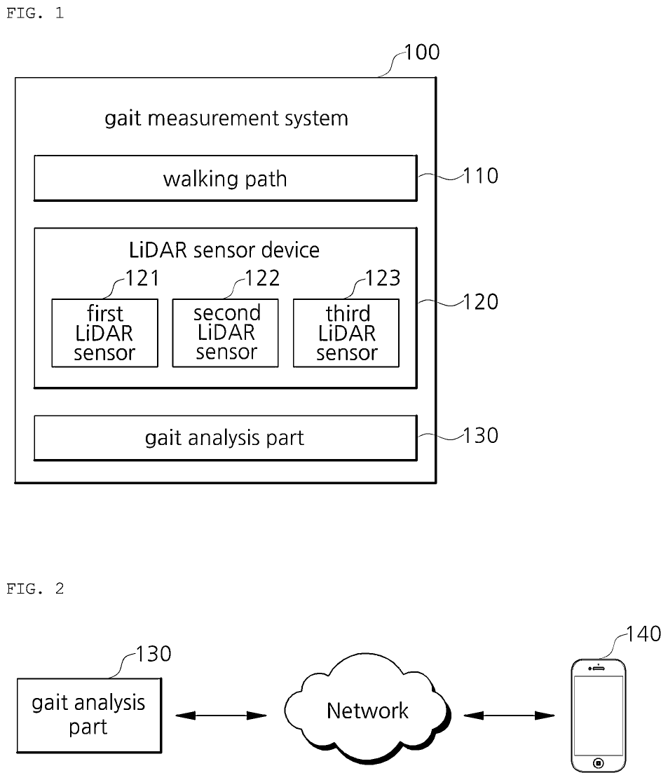

FIG. 2 is a diagram illustrating communication with a client terminal 140 in a gait measurement system 100 using multiple LiDARs at different heights according to an embodiment of the present disclosure. As shown in FIG. 2, according to an embodiment of the present disclosure, the gait measurement system 100 using the multiple LiDARs at different heights may further include the client terminal 140 on which an application program for outputting an analysis result obtained by the gait analysis part 130 is installed.

That is, as shown in FIG. 2, the analysis result obtained by the gait analysis part 130 may be transmitted to the client terminal 140 over a network, and the client terminal 140 may output the analysis result through the application program. Herein, the user of the client terminal 140 may be the measurement subject himself or herself or the measurement subject's guardian, or may be a medical worker or a manager of the gait measurement system 100.

In the meantime, the client terminal 140 may be a portable terminal or a computer. Herein, the portable terminal is a wireless communication device with portability and mobility secured. Examples of the portable terminal may include any types of handheld wireless communication devices, such as personal communication system (PCS), Global System for Mobile communications (GSM), Personal Digital Cellular (PDC), Personal Handy-phone System (PHS), personal digital assistant (PDA), International Mobile Telecommunications (IMT)-2000, code-division multiple access (CDMA)-2000, wideband code-division multiple access (W-CDMA), and wireless broadband Internet (WiBro) terminals. Examples of the computer may include a desktop computer, a notebook computer, and a laptop computer with a web browser.

In addition, the network may be a wired network, such as a local area network (LAN), a wide area network (WAN), or a value-added network (VAN), or any types of wireless networks, such as a mobile radio communication network, a satellite network, Bluetooth, wireless broadband Internet (WiBro), High Speed Downlink Packet Access (HSDPA), long-term evolution (LTE), and 3/4/5/6th-generation mobile telecommunication (3/4/5/6G).

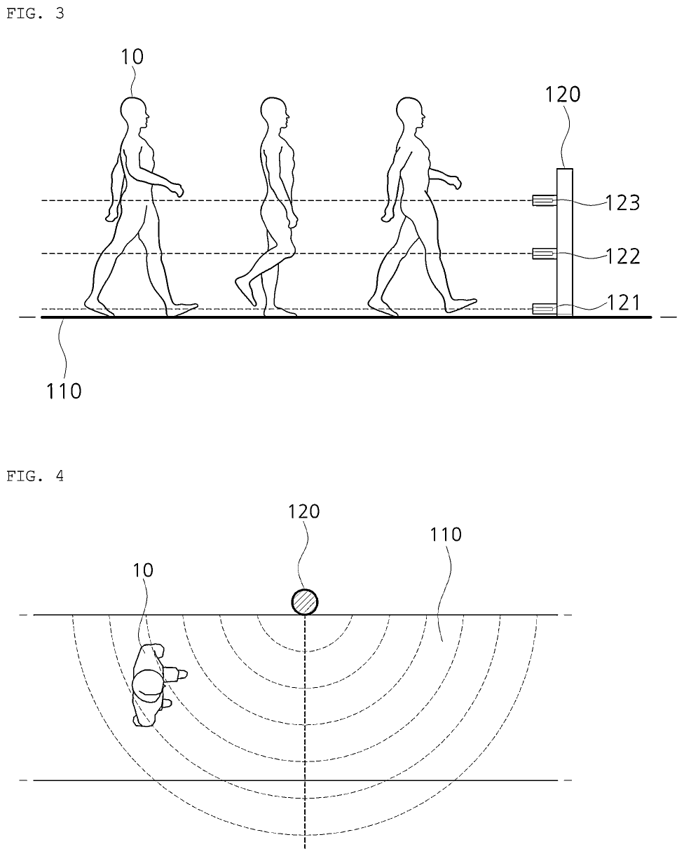

FIGS. 3 and 4 are diagrams illustrating gait measurement through a gait measurement system 100 using multiple LiDARs at different heights according to an embodiment of the present disclosure. Hereinafter, each element of a gait measurement system 100 using multiple LiDARs at different heights according to an embodiment of the present disclosure will be described in detail with reference to FIGS. 1 to 4.

The walking path 110 may be a floor surface on which the measurement subject 10 walks. Herein, the walking path 110 may have a width and length sufficient for the measurement subject 10 to walk comfortably, and may be realized in any place, as it can serve as a walking path 110 even in a narrow space, such as a passage of a building, as long as a walking distance is sufficient.

Depending on an embodiment, an indicative line or an arrow indicating the walking direction or walking course for the measurement subject 10 may be marked on the walking path 110. However, when the walking course is explicitly marked, the measurement subject 10 tries to walk according to the walking course, which may result in an intentional gait rather than a natural gait. Therefore, in order to obtain natural walking data, the walking direction or course may be marked with an arrow or an area in the longitudinal direction of the walking path 110 that is not a straight line and has a width ranging from about 20 to 60 cm, which is human shoulder width.

The LiDAR sensor device 120 may be installed at a side of the walking path 110 to scan the direction of the walking path 110. As shown in FIGS. 3 and 4, the LiDAR sensor device 120 is installed at a side, such as one end or side, of the walking path 110 rather than in the middle of the walking path 110 so that the LiDAR sensor device 120 does not interfere with the gait of the measurement subject 10 and a scanning area covers the entirety of the walking path 110.

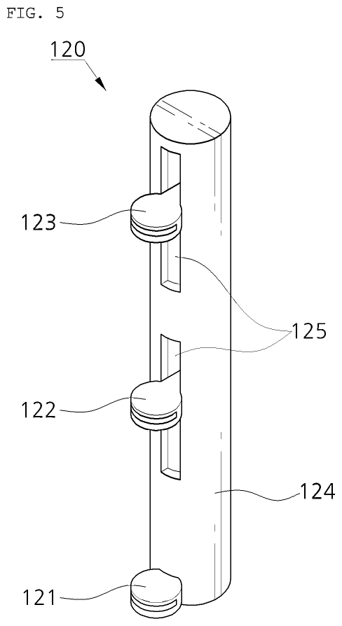

FIG. 5 is a diagram illustrating a LiDAR sensor device 120 of a gait measurement system 100 using multiple LiDARs at different heights according to an embodiment of the present disclosure. As shown in FIG. 5, according to an embodiment of the present disclosure, the LiDAR sensor device 120 of the gait measurement system 100 using the multiple LiDARs at different heights may include a first LiDAR sensor 121, a second LiDAR sensor 122, and a third LiDAR sensor 123. The LiDAR sensor device 120 may further include a body 124 on which the three LiDAR sensors 121, 122, and 123 are installed, and height adjusters 125.

Herein, the LiDAR sensors 121, 122, and 123 may scan the walking path 110, and scan a plane at a predetermined height above the floor. The first LiDAR sensor 121, the second LiDAR sensor 122, and the third LiDAR sensor 123 may scan planes at different heights to detect different parts of the body of the measurement subject 10.

More specifically, the first LiDAR sensor 121 may collect first scanning data by scanning, on the floor at a side of the walking path 110, a plane at foot height of the measurement subject 10 walking on the walking path 110. That is, the first LiDAR sensor 121 is installed on or close to the floor to scan the plane at foot height of the measurement subject 10, thereby detecting the foot positions, foot shapes, and foot directions of the measurement subject 10. The laser scanning height of the first LiDAR sensor 121 may range from about 3 to 10 cm, more specifically, 5 cm, above the floor. That is, the first LiDAR sensor 121 may generate the first scanning data by scanning, with lasers, the plane parallel to the floor and at the height of 3 to 10 cm above the floor.

The second LiDAR sensor 122 may collect second scanning data by scanning, at the side of the walking path 110, a plane at knee height of the measurement subject 10 walking on the walking path 110. That is, the second LiDAR sensor 122 is installed in the middle of the body 124 to scan the plane at knee height of the measurement subject 10, thereby detecting the knee positions of the measurement subject 10 and the directions the knees are facing. The laser scanning height of the second LiDAR sensor 122 may be the height of 20 to 50 cm above the floor, and the height of the second LiDAR sensor 122 may be adjustable through the height adjuster 125, which will be described in detail below.

The third LiDAR sensor 123 may collect third scanning data by scanning, at the side of the walking path 110, a plane at torso height of the measurement subject 10 walking on the walking path 110. That is, the third LiDAR sensor 123 is installed at an upper portion of the body 124 to scan the plane at torso height between the knee and the shoulder of the measurement subject 10, thereby detecting the torso position of the measurement subject 10 and the direction the torso is facing. In particular, the third LiDAR sensor 123 may perform scanning at pelvis height of the measurement subject 10 to recognize pelvic distortion from the position and direction of the pelvis. The laser scanning height of the third LiDAR sensor 123 may be the height of 50 to 100 cm above the floor, and the height of the third LiDAR sensor 123 may be adjustable through the height adjuster 125, which will be described in detail below.

The body 124 is a frame at which the three LiDAR sensors 121, 122, and 123 are installed, and may be installed upright on the floor. At the body 124, the three LiDAR sensors 121, 122, and 123 are arranged vertically. The body may have a power line or a power supply for supplying power to the LiDAR sensors 121, 122, and 123 built-in. The front side of the body may include the height adjusters 125, which will be described in detail below. In addition, the LiDAR sensor device 120 may be fixed and installed. However, in order for the LiDAR sensor device 120 to be moved and installed as needed, the bottom of the body 124 may include a plurality of wheels (not shown) for movement and installation. The body 124 has a cylindrical shape as shown in FIG. 5, but may be implemented in various shapes according to an embodiment.

The height adjusters 125 are to adjust the heights of the second LiDAR sensor 122 and the third LiDAR sensor 123, and may be rails. The height adjusters 125 are used to adjust the heights of the second LiDAR sensor 122 and the third LiDAR sensor 123 according to the heights of the knees and pelvis of the measurement subject 10. Accordingly, gaits of measurement subjects 10, such as adults or children, having different heights may be accurately measured. As shown in FIG. 5, the height adjuster 125 for the second LiDAR sensor 122 and the height adjuster 125 for the third LiDAR sensor 123 are provided separately. However, depending on an embodiment, one height adjuster 125 may be provided to extend the height adjustment range of the two LiDAR sensors 122 and 123.

The gait analysis part 130 may use signals detected by the LiDAR sensor device 120 to analyze the gait of the measurement subject 10 walking on the walking path 110. First, the gait analysis part 130 analyzes the first scanning data to specify the foot positions of the measurement subject 10, analyzes the second scanning data to specify the knee positions of the measurement subject 10, and analyzes the third scanning data to specify the torso position of the measurement subject 10. The gait analysis part 130 uses the foot positions of the measurement subject 10 to analyze at least one selected from the group of step length, the number of steps, and gait speed. The gait analysis part 130 uses the knee positions of the measurement subject 10 to analyze at least one selected from the group of the distance between the knees and knee movement speed. In addition, the gait analysis part 130 may combine the specified foot positions, knee positions, and torso position of the measurement subject 10 to analyze at least one selected from the group of body distortion and gait instability while the measurement subject 10 is walking.

More specifically, the gait analysis part 130 may use the first scanning data and the specified foot positions to analyze at least one selected from the group of step length, the number of steps, and gait speed. In addition, the positions of the left foot and the right foot and the respective step distances of the left foot and the right foot may be used to analyze left and right gait balance. Depending on an embodiment, the gait analysis part 130 may recognize the angles of the feet of the measurement subject 10 from the scanning data.

In addition, the gait analysis part 130 may perform an analysis by combining the specified foot, knee, and torso positions and the shapes of the feet, knees, and torso from the first scanning data, second scanning data, and third scanning data. The degree of distortion of the torso center, knees, and feet may be analyzed by combining pieces of information (positions, shapes, and directions) on the feet and knees, on the knees and torso, on the feet and torso, and on the feet, knees, and torso. In addition, the positions of the torso, knees, and feet are combined to analyze the movement instability of the measurement subject body's center of gravity. In this way, by analyzing the movement of the torso, knees, and feet during walking, information, such as the movement of the measurement subject body's center of gravity, required for rehabilitation medicine may be provided, and may be applied to the cases of the elderly walking with a cane, scoliosis, and changes in prognosis before and after surgery.

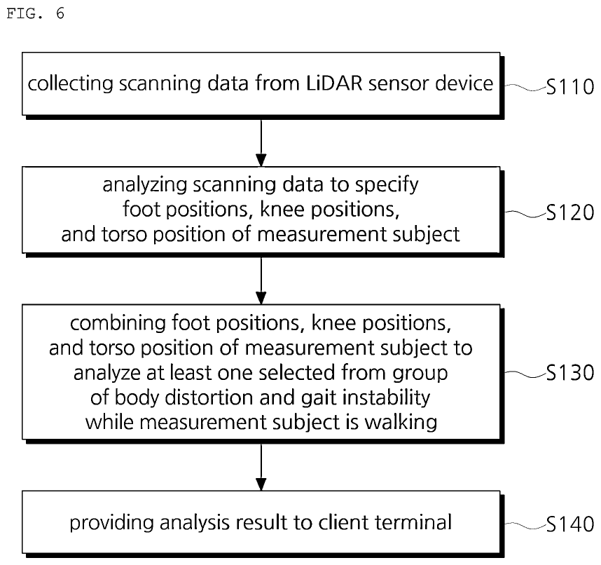

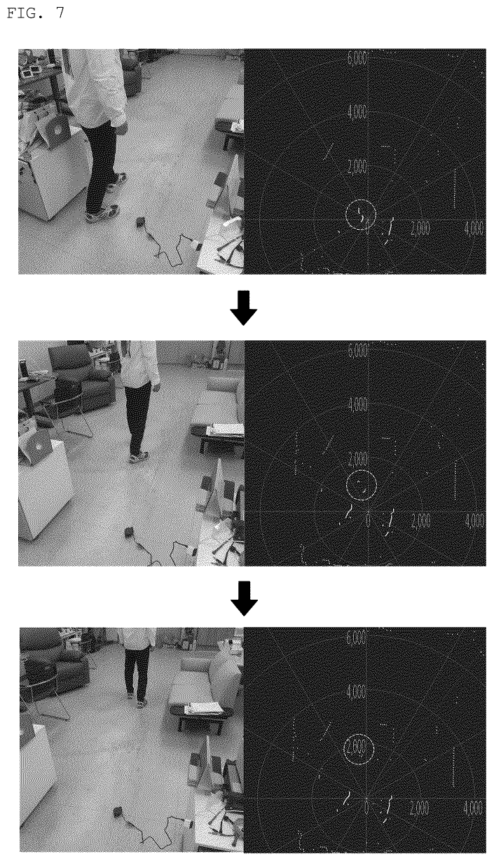

FIG. 6 is a diagram illustrating the flow of a gait measurement method using multiple LiDARs at different heights according to an embodiment of the present disclosure. As shown in FIG. 6, according to an embodiment of the present disclosure, a gait measurement method using multiple LiDARs at different heights is a gait measurement method of measuring a gait of a measurement subject 10 walking on a walking path 110. Each step of the gait measurement method is performed in a gait measurement system 100 including: a walking path 110 for a measurement subject 10 to walk; a LiDAR sensor device 120 installed at a side of the walking path 110 and configured to scan the direction of the walking path 110; and a gait analysis part 130 configured to use signals detected by the LiDAR sensor device 120 to analyze a gait of the measurement subject 10 walking on the walking path 110. The gait measurement method may include: collecting scanning data from the LiDAR sensor device 120 in step S110; analyzing the scanning data to specify foot positions, knee positions, and a torso position of the measurement subject 10 in step S120; and combining the foot positions, knee positions, and torso position of the measurement subject 10 to analyze at least one selected from the group of body distortion and gait instability while the measurement subject 10 is walking in step S130. The gait measurement method may further include providing an analysis result to a client terminal 140 in step S140.

In step S110, the scanning data obtained by the LiDAR sensor device 120 scanning the walking path 110 on which the measurement subject 10 is walking may be collected. Specifically, first scanning data may be collected by a first LiDAR sensor 121 scanning, on the floor at the side of the walking path 110, a plane at foot height of the measurement subject 10 walking on the walking path 110. Second scanning data may be collected by the second LiDAR sensor 122 scanning, at the side of the walking path 110, a plane at knee height of the measurement subject 10 walking on the walking path 110. Third scanning data may be collected by the third LiDAR sensor 123 scanning, at the side of the walking path 110, a plane at torso height of the measurement subject 10 walking on the walking path 110. That is, in the gait measurement environment as shown in FIG. 3 or 4, the first scanning data, second scanning data, and third scanning data collected by the LiDAR sensor device 120 when the measurement subject 10 is walking on the walking path 110 may be collected.

In step S120, the first scanning data is analyzed to specify the foot positions of the measurement subject 10, the second scanning data is analyzed to specify the knee positions of the measurement subject 10, and the third scanning data is analyzed to specify the torso position of the measurement subject 10. Herein, the positions of the feet, knees, and torso of the measurement subject 10 are specified from the first, second, and third scanning data and analyzed in synchronization with the measurement time. The positions, shapes, and facing angles of the feet, knees, and torso may be recognized.

In step S130, the specified foot positions, knee positions, and torso position of the measurement subject 10 are combined to analyze at least one selected from the group of body distortion and gait instability while the measurement subject 10 is walking. That is, in addition to step length, the number of steps, and gait speed, left and right gait balance, body distortion, pelvic distortion, and gait instability during walking may be analyzed. In step S130, the step length, the number of steps, the gait speed, the left and right gait balance, the body distortion, the pelvic distortion, and the gait instability during walking may be processed into a form, such as a score or graph, which enables intuitive user understanding.

In step S140, the analysis result obtained in step S130 may be provided to the client terminal 140 on which an application program for outputting the analysis result obtained by the gait analysis part 130 is installed. That is, the gait analysis result, such as the step length, the number of steps, the gait speed, the left and right gait balance, the body distortion, the pelvic distortion, and the gait instability during walking, may be provided to the client terminal 140 over the network. The measurement subject 10 himself or herself or a guardian, a medical worker, or a manager may check the gait analysis result through the client terminal 140, and use the gait analysis result for understanding the gait habits, recognizing body imbalance, correcting the gait, analyzing rehabilitation effects, or recognizing changes in prognosis before and after surgery.

FIG. 7 is a diagram illustrating gait measurement experiment and scanning data from a gait measurement system 100 using multiple LiDARs at different heights according to an embodiment of the present disclosure. The left of FIG. 7 is photographs of gait measurement, and the right of FIG. 7 is images showing the foot positions of the measurement subject 10 in the scanning data. The gait measurement system 100 using multiple LiDARs at different heights according to an embodiment of the present disclosure recognizes the foot positions of the measurement subject 10 accurately even in a narrow space as shown in the left photographs of the gait. As shown in the canning data of the LiDAR sensor device 120 in the right of FIG. 7, the gait analysis part 130 may specify the foot positions (indicated by the yellow dashed circle) of the measurement subject 10, and analyze changes in the foot positions to analyze step length, the number of steps, and gait speed. In the gait measurement system 100 using multiple LiDARs at different heights according to an embodiment of the present disclosure, these LiDAR sensors are arranged at different heights to analyze the positions and directions of the knees and torso in addition to the feet of the measurement subject 10.

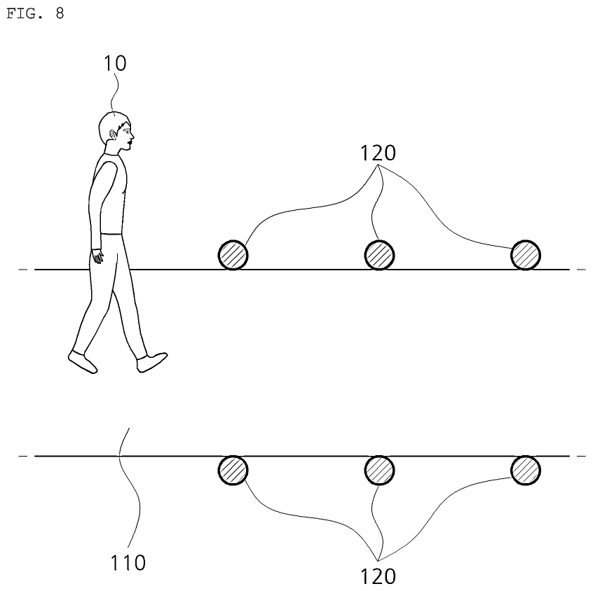

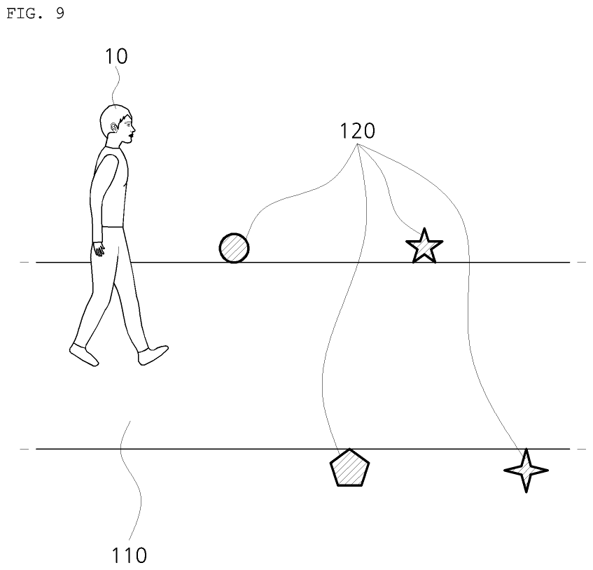

FIG. 8 is a diagram illustrating a plurality of LiDAR sensor devices 120 arranged in a gait measurement system 100 using multiple LiDARs at different heights according to an embodiment of the present disclosure. As shown in FIG. 8, in the gait measurement system 100 using multiple LiDARs at different heights according to an embodiment of the present disclosure, the LiDAR sensor devices 120 may be installed at intervals of a predetermined distance on the floor at a side of the walking path 110. According to the characteristics of a LiDAR sensor, the closer a subject is to the sensor, the more accurately the subject is recognized due to higher precision of the sensor in azimuth. Therefore, as shown in FIG. 8, the use of the plurality of LiDAR sensor devices 120 enables the positions, shapes, and directions of the feet, knees, and torso of the measurement subject 10 to be more precisely recognized.

Depending on an embodiment, the LiDAR sensor devices 120 may be arranged to face each other across the walking path 110. By arranging the LiDAR sensor devices 120 facing to each other, the directions (angles) the feet, knees, and torso of the measurement subject 10 are facing and the shapes thereof are more accurately recognized, so that complex gait characteristics, such as pelvic distortion or an out-toed gait, may be well analyzed.

FIG. 9 is a diagram illustrating a plurality of LiDAR sensor devices 120 of different shapes arranged in a gait measurement system 100 using multiple LiDARs at different heights according to an embodiment of the present disclosure. As shown in FIG. 9, in the gait measurement system 100 using multiple LiDARs at different heights according to an embodiment of the present disclosure, when there are a plurality of LiDAR sensor devices 120, the cross-sectional shapes thereof cut by the scanning plane may be different from each other to enable the LiDAR sensor devices 120 to be specified from scanning data of the different LiDAR sensor devices 120. Therefore, when a plurality of LiDAR sensor devices 120 are used, bodies 124 of different cross-sectional shapes may be used as shown in FIG. 9. When a LiDAR sensor device 120 of a circular shape recognizes a star or pentagonal shape, it is easily understood that the recognized object is another LiDAR sensor device 120. In this way, a plurality of LiDAR sensor devices 120 understand each other's locations from their shapes, so that a sensor recognizes the coordinates of the other sensors and the distances to the other sensors and scanning data is accurately mapped. Thus, through accurate mapping of a plurality of pieces of scanning data, the coordinates of the foot, knee, and torso positions and the angles and shapes of the feet, knees, and torso may be specified with high accuracy.

In the meantime, the gait analysis part 130 may predict a type of a disease from a gait analysis result by using an artificial intelligence model. The artificial intelligence model is trained using step length, the number of steps, gait speed, left and right gait balance, foot angles, and foot-knee-torso relative position information as input and types of diseases as output. That is, the step length, the number of steps, the gait speed, the left and right gait balance, the foot angles, and the foot-knee-torso relative position information that are the gait analysis result of the measurement subject 10 analyzed by the gait analysis part 130 are input to the artificial intelligence model, and a type of a predicted disease, such as musculoskeletal disorders, vestibular disorders, Parkinson's disease, or dementia, may be obtained as output. The output value may be provided to the client terminal 140 of the medical worker to assist in the medical worker's judgment.

Herein, a learning algorithm of the artificial intelligence model may be any one of the following: random forest, convolutional neural network (CNN), a recurrent neural network (RNN), and a transformer. Alternatively, an ensemble algorithm that is a combination of two or more algorithms may be used.

Depending on an embodiment, transfer learning may be used. Transfer learning is the reuse of a pre-trained model for a new problem. Since an already trained model is used, a neural network is deeply trained with relatively little data. In addition, it is also useful because most real problems do not typically have millions of labeled data to train complex models. In the present disclosure, transfer learning may be used to generate, from the artificial intelligence model pre-trained with a large amount of data, group-specific artificial intelligence models for measurement subjects 10 grouped by sex or age, thereby predicting a type of a disease more minutely and accurately.

As described above, according to a gait measurement system 100 and method using multiple LiDARs at different heights proposed in the present disclosure, a LiDAR sensor device 120 including a first LiDAR sensor for scanning at foot height, a second LiDAR sensor 122 for scanning at knee height, and a third LiDAR sensor 123 for scanning at torso height is installed at a side of a walking path 110, and signals detected by the LiDAR sensor device 120 are used to analyze a gait of a measurement subject 10 walking on the walking path 110. Accordingly, it is easy to recognize the movement behavior of the feet, knees, torso during walking. In addition, through an analysis by combining scanning data of the feet, knees, and torso during walking, information, such as the movement of the subject's center of gravity, required for rehabilitation medicine may be provided, and used for recognizing the use of a cane, scoliosis, or changes in prognosis before and after surgery.

In the meantime, the present disclosure may include a computer-readable recording medium including program instructions for performing operations implemented by various communication terminals. Examples of the computer-readable recording medium include magnetic recording media such as hard disks, floppy disks and magnetic tapes; optical data storage media such as CD-ROMs or DVD-ROMs; magneto-optical media such as floptical disks; and hardware devices, such as read-only memory (ROM), random-access memory (RAM), and flash memory, which are particularly structured to store and implement the program instructions.

The computer-readable recording medium may include program instructions, data files, data structures, and the like separately or in combinations. Herein, the program instructions being recorded in the computer-readable medium may correspond to program instructions that are specifically designed and configured for the embodiments of the present disclosure, or may correspond to program instructions that are disclosed and available to anyone skilled in the art of computer software. For example, the program commands may include machine language codes, which are created by a compiler, as well as high-level language codes, which may be executed by a computer by using an interpreter.

The foregoing description of the present disclosure is intended for exemplifications, and it will be understood by those skilled in the art that the present disclosure may be easily modified in other specific forms without changing the technical idea or essential features of the present disclosure. Therefore, it should be understood that the embodiments described above are illustrative in all aspects as and not restrictive. For example, each element described as a single type may be implemented in a dispersed form, and likewise elements described as dispersed may be implemented in a combined form.

The scope of the present disclosure is characterized by the appended claims rather than the detailed description described above, and it should be construed that all alterations or modifications derived from the meaning and scope of the appended claims and the equivalents thereof fall within the scope of the present disclosure.

What is claimed is:

1. A gait measurement system (100) using multiple LiDARs at different heights, the gait measurement system comprising:
a walking path (110) for a measurement subject (10) to walk;
a LiDAR sensor device (120) installed at a side of the walking path (110) and configured to scan a direction of the walking path (110); and
a gait analysis part (130) configured to use signals detected by the LiDAR sensor device (120) to analyze a gait of the measurement subject (10) walking on the walking path (110),
wherein the LiDAR sensor device (120) comprises:
a first LiDAR sensor (121) configured to collect first scanning data by scanning, on a floor at the side of the walking path (110), a plane at foot height of the measurement subject (10) walking on the walking path (110);
a second LiDAR sensor (122) configured to collect second scanning data by scanning, at the side of the walking path (110), a plane at knee height of the measurement subject (10) walking on the walking path (110); and
a third LiDAR sensor (123) configured to collect third scanning data by scanning, at the side of the walking path (110), a plane at torso height of the measurement subject (10) walking on the walking path (110).

2. The gait measurement system of claim 1, wherein the gait analysis part (130) is configured to
analyze the first scanning data to specify foot positions of the measurement subject (10), analyze the second scanning data to specify knee positions of the measurement subject (10), and analyze the third scanning data to specify a torso position of the measurement subject (10), and
use the foot positions of the measurement subject (10) to analyze at least one selected from a group of step length, the number of steps, and gait speed, and use the knee positions of the measurement subject (10) to analyze at least one selected from a group of a distance between knees and knee movement speed.

3. The gait measurement system of claim 2, wherein the gait analysis part (130) is configured to combine the specified foot positions, knee positions, and torso position of the measurement subject (10) to analyze at least one selected from a group of body distortion and gait instability while the measurement subject (10) is walking.

4. The gait measurement system of claim 1, wherein a plurality of the LiDAR sensor devices (120) are installed at intervals of a predetermined distance on the floor at the side of the walking path (110).

5. The gait measurement system of claim 1, wherein a plurality of the LiDAR sensor devices (120) are arranged to face each other across the walking path (110).

6. The gait measurement system of claim 1, further comprising
a client terminal (140) on which an application program for outputting an analysis result obtained by the gait analysis part (130) is installed.

7. A gait measurement method using multiple LiDARs at different heights to measure a gait of a measurement subject (10) walking on a walking path (110), the gait measurement method comprising the steps, each performed by a gait measurement system (100) including the walking path (110) for the measurement subject (10) to walk, a LiDAR sensor device (120) installed at a side of the walking path (110) and configured to scan a direction of the walking path (110), and a gait analysis part (130) configured to use signals detected by the LiDAR sensor device (120) to analyze the gait of the measurement subject (10) walking on the walking path (110), of:
(1) collecting scanning data obtained by the LiDAR sensor device (120) scanning the walking path (110) on which the measurement subject (10) is walking, wherein first scanning data is collected by a first LiDAR sensor (121) scanning, on a floor at the side of the walking path (110), a plane at foot height of the measurement subject (10) walking on the walking path (110), second scanning data is collected by a second LiDAR sensor (122) scanning, at the side of the walking path (110), a plane at knee height of the measurement subject (10) walking on the walking path (110), and third scanning data is collected by a third LiDAR sensor (123) scanning, at the side of the walking path (110), a plane at torso height of the measurement subject (10) walking on the walking path (110);
(2) analyzing the first scanning data to specify foot positions of the measurement subject (10), analyzing the second scanning data to specify knee positions of the measurement subject (10), and analyzing the third scanning data to specify a torso position of the measurement subject (10); and
(3) combining the specified foot positions, knee positions, and torso position of the measurement subject (10) to analyze at least one selected from a group of body distortion and gait instability while the measurement subject (10) is walking.

8. The gait measurement method of claim 7, further comprising
after the step (3), (4) providing an analysis result obtained in the step (3) to a client terminal (140) on which an application program for outputting the analysis result obtained by the gait analysis part (130) is installed.
